(19) United States
(12) Patent Application Publication
(10) Pub. No.: US 2024/0076350 A1
(43) Pub. Date: Mar. 7, 2024

(54) MHC: PEPTIDE COMPLEXES

(71) Applicant: Oxford University Innovation Limited, Oxford (GB)

(72) Inventors: Andrew MCMICHAEL, Oxford (GB); Geraldine GILLESPIE, Oxford (GB); Max QUASTEL, Oxford (GB)

(21) Appl. No.: 18/270,238

(22) PCT Filed: Dec. 31, 2021
(86) PCT No.: PCT/GB2021/053452

§ 371 Date: Jun. 29, 2023

(30) Foreign Application Priority Data
Dec. 31, 2020 (GB) 2020879.9
Jun. 1, 2021 (GB) 2107820.9

(51) Int. Cl. C07K 14/74

(52) U.S. Cl. CPC C07K 14/70539

(57) ABSTRACT

The invention relates to a mutant HLA-E heavy chain comprising one or more mutation which permits the formation of a HLA-E:peptide complex with increased stability when compared to the complex without the mutant HLA-E heavy chain. The invention also relates to a peptide which is capable of being crosslinked to the mutant HLA-E heavy chain, and a protein complex comprising or consisting of the mutant HLA-E heavy chain and peptide.

### FIELD

The present invention relates to HLA-E:peptide complexes, and in particular to stabilised HLA-E:peptide complexes for use in the identification of antigen binding polypeptides, such as antibodies and T-cell receptors.

### BACKGROUND

HLA-E is a non-polymorphic HLA class I molecule. There are two major alleles in the population differing only in one amino acid at position 107 which is outside the peptide binding groove (Strong et al., Correlating differential expression, peptide affinities, crystal structures, and thermal stabilities. J Biol Chem. 2003; 278(7):5082-90). The primary function of HLA-E is to bind a peptide usually termed ‘VL9’ which is derived from the signal peptide of classical HLA class I A, B, C molecules and HLA-G, but not HLA-E. The peptide has the sequence VMAPRTLVL, VMAPRTVLL, VMAPRTLLL, VMAPRTLIL, or VMAPRTLFL. The HLA-VL9 complex in turn binds to the NKG2A-CD94 inhibitory or NLG2C-CD94 activating receptors on natural killer cells and a subset of T cells (Braud et al., HLA-E binds to natural killer cell receptors CD94/NKG2A, B and C. Nature. 1998; 391(6669):795-9).

Other peptides derived from ‘self’ proteins, which may be abnormally expressed or mutated in cancer cells, or peptides derived from viruses or bacteria can also bind to HLA-E, but the large majority do so with lower binding affinity so cannot compete effectively with the VL9 peptide (Walters et al., Detailed and atypical HLA-E peptide binding motifs revealed by a novel peptide exchange binding assay. Eur J Immunol. 2020). However, in certain circumstances, presentation of the VL9 peptide is disturbed, for instance in cytomegalovirus (CMV) infection, in mycobacterial infection or in cancer cells, leading to HLA-E bound to other peptides being presented on the cell surface. These cells can then be recognised by CD8+ T cells, and a response initiated through their classical Major Histocompatibility complex class I (MHC-I)-restricted T cell receptor (TCR). As HLA-E is non-polymorphic and HLA-E restricted responses to pathogens have thus far been poorly characterised, further identification of T cell responses to peptide antigen bound to HLA-E may be useful for immunotherapies which could be applicable universally in the population because of the lack of HLA-E genetic polymorphism. For example, TCRs and/or monoclonal antibodies specific for HLA-E in complex with a peptide antigen could be generated and used therapeutically as cytotoxic reagents, or such antibodies and TCRs could manipulated as receptors, including chimeric receptors, which are transfected or transduced into effector cells to induce immune responses against the peptide antigen. Therefore, the generation of antibodies or T cells which recognise HLA-E bound to peptide antigens derived from a cancer, pathogen or even autoantigens has considerable therapeutic potential.

However, because B and T lymphocytes expressing such antibodies and TCRs are rarely stimulated naturally, it is necessary to identify peptides that bind to HLA-E, then identify whether such HLA-E:peptide complexes can induce an immune response, such as a CD8+ T cell response or a B lymphocyte-mediated antibody response, in vitro or in vivo in humans or in animal models, and then test whether they are presented on pathogenic cells.

For HLA-E:peptide specific antibody and T cell identification, validation, selection and immunisation, it is beneficial to use stable protein complexes, which may be soluble protein or prepared as multimers. In addition, cells transfected with DNA encoding single chain trimers of peptide linked to β2 microglobulin (β2m or beta2 microglobulin) linked to HLA-E heavy chain can be tested for stable display on the cell surface. As many peptides bind to HLA-E relatively weakly, the number of epitope peptides that it is possible to find and validate is relatively low without further stabilising the HLA-E:peptide complex.

If the binding of low affinity peptides could be stabilised, the number that could be used would be significantly increased. Fremont et al (Structural engineering of pMHC reagents for T cell vaccines and diagnostics, Chem Biol, 2007 August; 14(8):909-22) investigated improving the strength of the interaction between a peptide and an MHC class 1 molecule. This was achieved by adding a glycine and cysteine to the carboxy terminus of a nonamer epitope peptide, and mutating an amino acid in the MHC class I molecule (H-2Db) to a cysteine, replacing tyrosine at position 84, which normally closes the groove, and then linking the peptide and MHC class 1 molecule by a disulphide bridge between the inserted cysteines. However, as discussed later, this method, whilst stabilising the interaction, resulted in unwanted conformational changes to the natural HLA-E:peptide complex.

There is therefore a need for an alternative and/or improved method of stabilising peptide binding to HLA-E, in which conformational identity is retained.

### SUMMARY OF THE INVENTION

### Mutant HLA-E

In a first aspect, there is provided a mutant HLA-E heavy chain comprising one or more mutation which permits the formation of a HLA-E:peptide complex with increased stability when compared to the complex without the mutant HLA-E heavy chain. The complex may further comprise β2 microglobulin.

The mutant HLA-E heavy chain may be capable of being crosslinked to a peptide antigen. The crosslinking may introduce a covalent bond between an amino acid in the mutant HLA-E heavy chain and an amino acid in the peptide antigen. A crosslink may be capable of being formed between residues in the HLA-E heavy chain.

The crosslinking may be via a disulphide bond between an amino acid in the mutant HLA-E heavy chain, and an amino acid in the peptide antigen. The crosslinking may be between one of the mutations in the HLA-E heavy chain and the peptide antigen. The crosslinking may be between two mutations in the HLA-E heavy chain.

The mutant HLA-E heavy chain may be derived from human HLA-E

(SEQ ID NO: 1)
(MVDGTLLLLLSEALALTQTWAGSHSLKYFHTSVSRPGRGEPRFISVGYV
DDTQFVRFDNDAASPRMVPRAPWMEQEGSEYWDRETRSARDTAQIFRVNL
RTLRGYYNQSEAGSHTLQWMHGCELGPDGRFLRGYEQFAYDGKDYLTLNE
DLRSWTAVDTAAQISEQKSNDASEAEHQRAYLEDTCVEWLHKYLEKGKET
LLHLEPPKTHVTHHPISDHEATLRCWALGFYPAEITLTWQQDGEGHTQDT
ELVETRPAGDGTFQKWAAVVVPSGEEQRYTCHVQHEGLPEPVTLRWKPAS
QPTIPIVGIIAGLVLLGSVVSGAVVAAVIWRKKSSGGKGGSYSKAEWSDS
AQGSESHSL)
or
SEQ ID NO: 2
(GSHSLKYFHTSVSRPGRGEPRFISVGYVDDTQFVRFDNDAASPRMVPRA
PWMEQEGSEYWDRETRSARDTAQIFRVNLRTLRGYYNQSEAGSHTLQWMH
GCELGPDGRFLRGYEQFAYDGKDYLTLNEDLRSWTAVDTAAQISEQKSND
ASEAEHQRAYLEDTCVEWLHKYLEKGKETLLHLEPPKTHVTHHPISDHEA
TLRCWALGFYPAEITLTWQQDGEGHTQDTELVETRPAGDGTFQKWAAVVV
PSGEEQRYTCHVQHEGLPEPVTLRWKPASQPTIPIVGIIAGLVLLGSVVS
GAVVAAVIWRKKSSGGKGGSYSKAEWSDSAQGSESHSL)
or
SEQ ID NO: 3
(MVDGTLLLLLSEALALTQTWAGSHSLKYFHTSVSRPGRGEPRFISVGYV
DDTQFVRFDNDAASPRMVPRAPWMEQEGSEYWDRETRSARDTAQIFRVNL
RTLRGYYNQSEAGSHTLQWMHGCELGPDRRFLRGYEQFAYDGKDYLTLNE
DLRSWTAVDTAAQISEQKSNDASEAEHQRAYLEDTCVEWLHKYLEKGKET
LLHLEPPKTHVTHHPISDHEATLRCWALGFYPAEITLTWQQDGEGHTQDT
ELVETRPAGDGTFQKWAAVVVPSGEEQRYTCHVQHEGLPEPVTLRWKPAS
QPTIPIVGIIAGLVLLGSVVSGAVVAAVIWRKKSSGGKGGSYSKAEWSDS
AQGSESHSL)
or
SEQ ID NO: 4
(GSHSLKYFHTSVSRPGRGEPRFISVGYVDDTQFVRFDNDAASPRMVPRA
PWMEQEGSEYWDRETRSARDTAQIFRVNLRTLRGYYNQSEAGSHTLQWMH
GCELGPDRRFLRGYEQFAYDGKDYLTLNEDLRSWTAVDTAAQISEQKSND
ASEAEHQRAYLEDTCVEWLHKYLEKGKETLLHLEPPKTHVTHHPISDHEA
TLRCWALGFYPAEITLTWQQDGEGHTQDTELVETRPAGDGTFQKWAAVVV
PSGEEQRYTCHVQHEGLPEPVTLRWKPASQPTIPIVGIIAGLVLLGSVVS
GAVVAAVIWRKKSSGGKGGSYSKAEWSDSAQGSESHSL).

The one or more mutation in the HLA-E heavy chain may be in the A pocket. The one or more mutation may be of an amino acid at one or more of position 28, 80, 84, 98, 184, 189, or 192 of SEQ ID NO: 1 or SEQ ID NO: 3, or one or more amino acid at a position equivalent thereto of SEQ ID NO: 1 or SEQ ID NO: 3 (these include the 21 amino acid signal peptide). The one or more mutation may be of the amino acids at positions 28 and 192 of SEQ ID NO: 1 or SEQ ID NO: 3, or the amino acids at positions equivalent thereto of SEQ ID NO: 1 or SEQ ID NO: 3.

Where the HLA-E heavy chain signal peptide is not present, the one or more mutation in the A pocket may be of an amino acid at one or more of position 7, 59, 63, 77, 163, 167, or 171 of SEQ ID NO: 2 or SEQ ID NO: 4, or one or more amino acid at a position equivalent thereto of SEQ ID NO: 2 or SEQ ID NO: 4. The one or more mutation may be of the amino acids at positions 7 and 171 of SEQ ID NO: 2 or SEQ ID NO: 4, or at positions equivalent thereto of SEQ ID NO:2 or SEQ ID NO: 4.

The one or more mutation in the HLA-E heavy chain may be in the B pocket. The one or more mutation may be of an amino acid at one or more of position 28, 30, 66, 84, 87, 88, or 91 of SEQ ID NO: 1 or SEQ ID NO: 3, or one or more amino acid at a position equivalent thereto of SEQ ID NO: 1 or SEQ ID NO: 3 (the sequences include the 21 amino acid signal peptide). The one or more mutation may be of the amino acid at position 66 of SEQ ID NO: 1 or SEQ ID NO: 3, or the amino acid at a position equivalent thereto of SEQ ID NO: 1 or SEQ ID NO: 3.

Where the HLA-E heavy chain signal peptide is not present, the one or more mutation in the B pocket may be of an amino acid at one or more of position 7, 9, 45, 63, 66, 67, or 70 of SEQ ID NO: 2 or SEQ ID NO: 4, or one or more amino acid at a position equivalent thereto of SEQ ID NO: 2 or SEQ ID NO: 4. The one or more mutation may be of the amino acid at position 45 of SEQ ID NO: 2 or SEQ ID NO: 4, or the amino acid at a position equivalent thereto of SEQ ID NO: 2 or SEQ ID NO: 4.

Where the HLA-E heavy chain signal peptide is not present, the one or more mutation may be of the amino acid at position 84 and at position 139 of SEQ ID NO: 2 or SEQ ID NO: 4, or one or more amino acid at a position equivalent thereto of SEQ ID NO: 2 or SEQ ID NO: 4. The mutation at position 84 or a position equivalent thereto may be to a cysteine, and the mutation at position 139 or a position equivalent thereto may be to a cysteine. This allows the formation of a disulphide bond between the cysteine at position 84 and the cysteine at position 139. This crosslink may improve the binding of a peptide of interest in the HLA-E:peptide complex, demonstrated by increased Tm (FIG. 7).

Where the HLA-E heavy chain signal peptide is not present, the one or more mutation may be of the serine at position 147 to a tryptophan of SEQ ID NO: 2 or SEQ ID NO: 4, or one or more amino acid at a position equivalent thereto of SEQ ID NO: 2 or SEQ ID NO: 4. The mutation at position 147 or a position equivalent thereto may be to a tryptophan. Such a crosslink closes the E-pocket of HLA-E, which is used by the signal peptide VL9 but which is not used by pathogen or cancer-derived peptides binding to HLA-E. Binding of the peptides is enhanced, demonstrated by increased melting temperature (Tm) of the protein (FIG. 8).

Where the HLA-E heavy chain signal peptide is not present, the one or more mutation may be of the histidine at position 99 of SEQ ID NO: 2 or SEQ ID NO: 4, or one or more amino acid at a position equivalent thereto of SEQ ID NO: 2 or SEQ ID NO: 4. The mutation at position 99 or a position equivalent thereto may be to a tyrosine.

Where the HLA-E heavy chain signal peptide is not present, the one or more mutation may be of the phenylalanine at position 116 of SEQ ID NO: 2 or SEQ ID NO: 4, or one or more amino acid at a position equivalent thereto of SEQ ID NO: 2 or SEQ ID NO: 4. The mutation at position 116 or a position equivalent thereto may be to a tyrosine.

The mutation of an amino acid at position 99, 116 or 147 of SEQ ID NO: 2 or SEQ ID NO: 4, or at an amino acid at a position equivalent thereto, closes/alters different HLA-E peptide binding pockets which are used optimally by the signal peptide VL9. This in contrast to many low affinity pathogen or cancer-derived peptides that bind to HLA-E. These mutations enhance the binding of the pathogen or cancer-derived peptides, demonstrated by increased melting temperature (Tm) of the protein (FIGS. 8 and 9).

The mutant HLA-E heavy chain may comprise or consist of the sequence of

SEQ ID NO: 5
(MVDGTLLLLLSEALALTQTWAGSHSLKYFHTSVSRPGRGEPRFISVGYV
DDTQFVRFDNDAASPRCVPRAPWMEQEGSEYWDRETRSARDTAQIFRVNL
RTLRGYYNQSEAGSHTLQWMHGCELGPDGRFLRGYEQFAYDGKDYLTLNE
DLRSWTAVDTAAQISEQKSNDASEAEHQRAYLEDTCVEWLHKYLEKGKET
LLHLEPPKTHVTHHPISDHEATLRCWALGFYPAEITLTWQQDGEGHTQDT
ELVETRPAGDGTFQKWAAVVVPSGEEQRYTCHVQHEGLPEPVTLRWKPAS
QPTIPIVGIIAGLVLLGSVVSGAVVAAVIWRKKSSGGKGGSYSKAEWSDS
AQGSESHSL)
or
SEQ ID NO: 6
(GSHSLKYFHTSVSRPGRGEPRFISVGYVDDTQFVRFDNDAASPRCVPRA
PWMEQEGSEYWDRETRSARDTAQIFRVNLRTLRGYYNQSEAGSHTLQWMH
GCELGPDGRFLRGYEQFAYDGKDYLTLNEDLRSWTAVDTAAQISEQKSND
ASEAEHQRAYLEDTCVEWLHKYLEKGKETLLHLEPPKTHVTHHPISDHEA
TLRCWALGFYPAEITLTWQQDGEGHTQDTELVETRPAGDGTFQKWAAVVV
PSGEEQRYTCHVQHEGLPEPVTLRWKPASQPTIPIVGIIAGLVLLGSVVS
GAVVAAVIWRKKSSGGKGGSYSKAEWSDSAQGSESHSL)
or
SEQ ID NO: 7
(MVDGTLLLLLSEALALTQTWAGSHSLKYFHTSVSRPGRGEPRFISVGYV
DDTQFVRFDNDAASPRCVPRAPWMEQEGSEYWDRETRSARDTAQIFRVNL
RTLRGYYNQSEAGSHTLQWMHGCELGPDRRFLRGYEQFAYDGKDYLTLNE
DLRSWTAVDTAAQISEQKSNDASEAEHQRAYLEDTCVEWLHKYLEKGKET
LLHLEPPKTHVTHHPISDHEATLRCWALGFYPAEITLTWQQDGEGHTQDT
ELVETRPAGDGTFQKWAAVVVPSGEEQRYTCHVQHEGLPEPVTLRWKPAS
QPTIPIVGIIAGLVLLGSVVSGAVVAAVIWRKKSSGGKGGSYSKAEWSDS
AQGSESHSL)
or
SEQ ID NO: 8
(GSHSLKYFHTSVSRPGRGEPRFISVGYVDDTQFVRFDNDAASPRCVPRA
PWMEQEGSEYWDRETRSARDTAQIFRVNLRTLRGYYNQSEAGSHTLQWMH
GCELGPDRRFLRGYEQFAYDGKDYLTLNEDLRSWTAVDTAAQISEQKSND
ASEAEHQRAYLEDTCVEWLHKYLEKGKETLLHLEPPKTHVTHHPISDHEA
TLRCWALGFYPAEITLTWQQDGEGHTQDTELVETRPAGDGTFQKWAAVVV
PSGEEQRYTCHVQHEGLPEPVTLRWKPASQPTIPIVGIIAGLVLLGSVVS
GAVVAAVIWRKKSSGGKGGSYSKAEWSDSAQGSESHSL).

The one or more mutation in the HLA-E heavy chain may be to one or more amino acid with a free sulphydryl group. The one or more mutation may be to one or more cysteine. The one or more mutation may be to one or more lysine. This may involve the use of an additional small molecule to bridge the amino-acids to be crosslinked

### Peptide

In a second aspect, there is provided a peptide which is capable of being crosslinked to the mutant HLA-E heavy chain of the first aspect. Alternatively or additionally, there is provided a peptide which is capable of forming an HLA-E:peptide complex with the mutant HLA-E heavy chain of the first aspect, wherein the complex has increased stability when compared to a complex of the peptide and a non-mutated HLA-E heavy chain.

Preferably the peptide is about 8 to about 12 amino acids long.

The crosslinking may introduce a covalent bond between an amino acid in the mutant HLA-E heavy chain and an amino acid in the peptide.

The amino acid crosslinked in the peptide may be a naturally occurring amino acid or synthetic amino acid.

The crosslinking may be via a disulphide bond between an amino acid in the mutant HLA-E heavy chain and an amino acid in the peptide.

The crosslinking of an amino acid in the peptide may be with an amino acid which is mutated in the HLA-E heavy chain, for example a cysteine which is introduced into the HLA-E heavy chain.

The peptide which binds to the mutant HLA-E heavy chain of the first aspect may be, or may be derived from, a peptide antigen, for example a peptide antigen which binds to HLA-E and is presented to a T-cell receptor (TCR). The peptide may be, or may be derived from, a naturally occurring peptide antigen which naturally binds to HLA-E and is presented to a TCR. The peptide may be, or may be derived from, VMAPRTLVL (SEQ ID NO: 9). The peptide may be, or may be derived from, RMYSPTSIL (SEQ ID NO: 10). The peptide may be, or may be derived from, any peptide antigen known to bind weakly to the HLA-E heavy chain, or to be an epitope recognised by a T lymphocyte. The naturally occurring peptide antigen may interact with an HLA-E heavy chain so weakly that the melting point (Tm) is undeterminable. The naturally occurring peptide antigen may comprise a methionine, leucine, glutamine, valine, isoleucine, phenylalanine at position 2.

In order for a peptide, such as a naturally occurring peptide antigen, to bind to the HLA-E heavy chain of the first aspect of the invention with increased stability, the peptide may have a mutation at the amino acid in the first or second position. The mutation may be a substitution and/or addition.

The peptide may be extended by two or more amino acids at its N-terminus or C-terminus, and at least one of the amino acids in the extended portion may have an amino acid, naturally occurring or synthetic, suitable for crosslinking to the mutant HLA-E heavy chain.

The mutation may be to a cysteine. The mutation may be homocysteine.

The mutation may be to a synthetic/non-natural amino acid. The synthetic amino acid may comprise a free sulphydryl group. The synthetic amino acid may be capable of forming a disulphide bond. The synthetic amino acid may be a homocysteine analogue. The synthetic amino acid may have a longer side chain than homocysteine ending with a sulphydryl group. The synthetic amino acid may be (2S)-2-amino-5-sulfanylpentanoic acid. The synthetic amino acid may be (2S)-2-amino-6-sulfanylhexanoic acid. The synthetic amino acid may be synthesized or may be available commercially.

### Complex

In a third aspect, there is provided a protein complex comprising or consisting of a mutant HLA-E heavy chain of the first aspect and a peptide of the second aspect. The mutant HLA-E heavy chain of the first aspect and a peptide of the second aspect may be crosslinked. The crosslink may be via a disulphide bond. The complex may further comprise β2 microglobulin. The crosslink may be between a mutant amino acid in the HLA-E heavy chain and the amino acid at the first or second position in the peptide. The mutation in the HLA-E heavy chain may be as described in the first aspect, and the amino acid at the first or second position of the peptide may be as described in the second aspect.

In a fourth aspect, there is provided a polypeptide comprising the sequence of a mutant HLA-E heavy chain of the first aspect, and further comprising the sequence of a peptide of the second aspect. The sequence of a peptide of the second may be separated from the sequence of C by a linker sequence. The polypeptide may also further comprise or consist of the sequence of β2 microglobulin. The sequence of β2 microglobulin may be separated from the sequence of a mutant HLE-E heavy chain of the first aspect and/or the sequence of a peptide of the second aspect by a linker sequence. Any linker sequence may be cleavable, such that the mutant HLA-E heavy chain, peptide and/or β2 microglobulin sequences may be separated and processed, such as folded, separately. The polypeptide may comprise, in order, a cleavable signal peptide, a peptide of the second aspect, a linker sequence, β2microglobulin, a linker sequence, a mutant HLE-E heavy chain of the first aspect.

Alternatively, the mutant HLA-E heavy chain and the β2microglobulin may be expressed separately, for example in bacterial cells such as E. coli or in a mammalian cell line, purified as soluble pure proteins and then mixed with the purified, preferably synthetic, peptide to refold in vitro. The complex may then be purified by size exclusion FPLC.

Alternatively, the soluble purified mutant HLA-E heavy chain and the β2microglobulin may be refolded with the ultraviolet light sensitive peptide VMAPJTLVL, where J is 3-amino-3-(2-nitrophenyl)-propionic acid, and then mixed with an excess of the purified, preferably synthetic, peptide in the presence of UV light. This will result in cleavage of the peptide, then peptide exchange with the cross linkable peptide. The product may then be purified by size exclusion FPLC.

The peptide in the complex may be expressed from a DNA construct or may be synthesised de novo.

### Nucleic Acid

In a fifth aspect, there is provided a nucleic acid encoding the mutant HLA-E heavy chain of the first aspect, a peptide of the second aspect, a complex of the third aspect, and/or a polypeptide of the fourth aspect.

The nucleic acid may be a DNA or RNA molecule. The DNA may be a cDNA.

In a sixth aspect, there is provided a vector comprising a nucleic acid of the fifth aspect. The vector may be an expression vector. The vector may be a plasmid. The vector may be a viral vector, such as a retroviral vector, lentiviral vector, an adenoviral or other viral vector. The vector may comprise nucleic acid disclosed herein, encoded in a single open reading frame, two distinct open reading frames encoding a mutant HLA-E heavy chain of the first aspect and a peptide of the second aspect respectively; or three distinct open reading frames encoding a mutant HLA-E heavy chain of the first aspect, a peptide of the second aspect and β2 microglobulin.

### Cells

In a seventh aspect, there is provided a cell or population of cells comprising and/or encoding one or more of a mutant HLA-E heavy chain of the first aspect, a peptide of the second aspect, a complex of the third aspect, a polypeptide of the fourth aspect, a nucleic acid of the fifth aspect, or a vector of the sixth aspect.

The cell or population of cells may harbour a first expression vector which comprises nucleic acid encoding a mutant HLA-E heavy chain of the first aspect, and a second expression vector comprising nucleic acid encoding a peptide of the second aspect. Alternatively, a peptide of the second aspect may be synthesised de novo and added to the cell or population of cells.

The cell or population of cells may harbour an expression vector which comprises nucleic acid encoding a complex of the third aspect, or a polypeptide of the fourth aspect.

The cell or population of cells may express or harbour one or more of, or all of, a mutant HLA-E heavy chain of the first aspect, a peptide of the second aspect, a complex of the third aspect a polypeptide of the fourth aspect, a nucleic acid of the fifth aspect, and/or a vector of the sixth aspect.

The cell may be any nucleated cell.

The cell or population of cells may be isolated and/or recombinant and/or non-naturally occurring and/or engineered.

### Method of Providing a Stabilised HLA-E:Peptide Complex

In an eighth aspect, there is provided a method of increasing the stability of an HLA-E:peptide complex, wherein the method comprises: crosslinking a peptide of the second aspect to a mutant HLA-E heavy chain of the first aspect, such that the HLA-E:peptide complex is stabilised, when compared to a complex of the peptide and a non-mutated HLA-E heavy chain.

Alternatively, the method comprises: contacting a mutant HLA-E of the first aspect with a peptide of the second aspect, such that the HLA-E:peptide complex is stabilised, when compared to a complex of the peptide and a non-mutated HLA-E heavy chain.

Alternatively, the method comprises: expressing or folding a mutant HLA-E of the first aspect in the presence of a peptide of the second aspect, such that the HLA-E:peptide complex is stabilised, when compared to a complex of the peptide and a non-mutated HLA-E heavy chain.

The mutant HLA-E may stabilise the binding of a peptide in the HLA-E:peptide complex when compared to a non-mutated HLA-E heavy chain molecule.

The crosslinking may be covalent, for example via disulphide bonding.

### Method of Identifying New Antibodies or TCRs which Recognise a HLA-E Bound Peptide

In a ninth aspect, there is provided a method of identifying antigen binding polypeptides which recognise a HLA-E bound peptide complex, wherein the method comprises:
a) crosslinking a peptide of the second aspect to a mutant HLA-E heavy chain of the first aspect, to form a crosslinked HLA-E:peptide complex; and
b) screening for antigen binding polypeptides which recognise the crosslinked HLA-E:peptide complex.

Alternatively, step (a) comprises: providing an HLA-E:peptide complex comprising a mutant HLA-E of the first aspect and a peptide of the second aspect.

The antigen binding polypeptide may be an antibody or a T-cell receptor (TCR).

The crosslinking may be covalent, for example via disulphide bonding.

The HLA-E-peptide complexes of the invention may be made into multimers and coupled to a fluorochrome or microbead, and then be used to select T lymphocytes that express receptors (TCRs) which recognise the complex, or B lymphocytes that express antibody receptors that can bind the complex. The selected cells can be cloned, and their TCRs or antibodies, respectively, purified. The nucleic acid encoding the TCRs or antibodies, or the polypeptide sequence of the TCRs or antibodies, can be isolated and used to generate the TCRs or antibodies as soluble proteins. The nucleic acid encoding the TCRs or antibodies may also be transfected or transduced into live cells which then express the receptors. There are a number of methods suitable for the transfection or transduction of cells with nucleic acid (such as DNA, cDNA or RNA) encoding the TCRs or antibodies (see for example Robbins et al., (2008) J Immunol. 180: 6116-6131).

The data presented herein demonstrate that the HLA-E heavy chain and peptide interaction can be modified to be crosslinked, and the complex can be stabilised whilst retaining the natural complex conformation, thus allowing the reliable identification of potential therapeutic antibodies and TCRs recognising the complex. Specifically, the invention provides an improved method of cross linking weak binding peptides to the HLA-E heavy chain that avoids manipulation of the C terminus region of the peptide-HLA-E complex, which appears to be sensitive to small structural changes, by focussing on the A and B pocket of the HLA-E heavy chain. The B pocket accommodates the side chain of the second amino acid in the peptide, and the A pocket binds the amino terminus of the peptide.

The invention thus permits the testing of expression gene libraries that express antibodies or fragments thereof, or T cell receptors or fragments thereof for potential therapeutic application. For example, stabilised HLA-E-peptide complexes, comprising an HLA-E heavy chain according to the invention and a peptide according to the invention, can be used to generate specific monoclonal antibodies. Antibodies which bind specifically to single HLA-E:peptide complexes have potential for use in the treatment of cancer and infections. They could be used alone, blocking interactions with T cells or natural killer cells or to recruit complement or other cells through their Fc regions. Such monoclonal antibodies could alternatively be developed as bi- or multi-specific soluble reagents, binding to the cancer or infected cell and recruiting other cell types such as effector T lymphocytes. They could also be developed as chimeric receptors and transfected or transduced into effector cells, activating these cells on binding to their ligands, through intracellular signalling domains added to the carboxy terminal end of the antibody. Similar therapeutic approaches could be developed for T cell receptors isolated from T lymphocytes selected with the stabilised HLA-E peptide complexes.

As used herein, “stabilisation” or “stability” refers to proteins or complexes of proteins and peptides that can be purified as a homogenous material with a distinct molecular mass and a thermal stability that is measurably greater than that of HLA-E-beta 2microglobulin complexes that lack bound peptide. Molecular stability and homogeneity can be measured, for example, by using blue Native gels analysis where HLA-E-β2m:peptide complexes may give a tight single band indicating high affinity binding, or a diffuse band indicating low affinity binding or a mix of the two (Walters et al Nat Comm 2018). Thermal melt analysis gives further information on the stability of the peptide binding to the HLA-E heavy chain.

Stronger binding peptides give distinct unfolding profiles as the temperature is raised, with higher temperatures for 50% unfolding indicating higher affinity binding, whereas weak binding peptides do not give a clear profile (FIG. 1). An increased stability, measured in these assays, may result from the covalent binding of the peptide to the HLA-E heavy chain, preferably whilst the conformation and antigenicity of the non-covalently bound peptide: HLA-E complex is maintained. This may then be tested by binding of specific monoclonal antibodies (FIG. 2) and/or recognition by antigen specific T cells.

As used herein, the terms “peptide” and peptide antigen may be used interchangeably.

As used herein, the term “crosslink” or “crosslinked” refers to a bond that links one polymer chain, such as a peptide or polypeptide, to another. Such crosslinks links may take the form of covalent bonds or ionic bonds and the polymers can be either synthetic polymers or natural polymers. The skilled person will appreciate that many types of crosslinks are possible, and that the literature can be consulted to identify suitable crosslinks and methodologies for introducing such crosslinks into peptides and polypeptides. In an embodiment the cross link is a disulphide bond.

Within the scope of the invention are phenotypically silent variants of any mutant HLA-E heavy chain disclosed herein. As used herein the term “phenotypically silent variants” is understood to refer to a protein which incorporates one or more further amino acid changes, in which a protein has a similar phenotype to the corresponding protein without said change(s). As is known to those skilled in the art, it may be possible to produce mutant HLA-E that incorporates changes outside of the peptide binding portion(s) compared to those detailed above without altering the stability of the complex formation, peptide binding affinity, and/or function. Such trivial variants are included in the scope of this invention. Those HLA-E heavy chains in which one or more conservative substitutions have been made also form part of this invention.

Mutagenesis can be carried out using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning—A Laboratory Manual (3rd Ed.) CSHL Press. Further information on ligation independent cloning (LIC) procedures can be found in Rashtchian, (1995) Curr Opin Biotechnol 6(1):30-6.

The skilled person will appreciate that preferred features of any one embodiment and/or aspect of the invention may be applied to all other embodiments and/or aspects of the invention.

The invention will now be described, by way of example only, by the following figures and examples.

### BRIEF DESCRIPTION OF THE DRAWINGS

FIG. 1—demonstrates the stability of HLA-E:peptide complexes by thermal melt determination. (A and B) HLA-E was refolded with the signal peptide VMAPRTLVL and the mycobacterial peptide RLPAPAKL, then heated (x axis) and binding of detector dye (y axis) shows unfolding. The vertical line indicates the temperature that unfolds 50% of the protein, Tm. (C) The HIV Gag peptide RMYSPTSIL (RL9) was used to refold HLA-E but a Tm could not be determined. (D)When the RL9 peptide C terminus was extended and the peptide was refolded with HLA-E with a tyrosine to cysteine mutation, (seen in E), a stable molecule was obtained that gave a measurable Tm.

FIG. 2—demonstrates that monoclonal antibodies that bind crosslinked HLA-E-RL9 fail to bind non-crosslinked RL9. HLA-E was expressed as a single chain trimer (SCT) of peptide-β2microglobulin-HLA-E heavy chain by DNA transfection into HEK293T cells. The peptide was either extended by addition of a glycine-cysteine at its C terminus and cross linked to a cysteine mutated from tyrosine at position 84 in the HLA-E heavy chain—numbered including the signal sequence—(Cross linked), or the peptide was simply extended by a glycine-serine linker to the amino terminus of β2m and with a tyrosine to alanine change at position 84 to open that end of the peptide binding groove (not cross-linked). The peptide was either the HLA class Ia signal peptide VMAPRTLVL, cross linked or not, or the HIV-1 Gag peptide RMYSPSTIL, cross linked or not. The antibodies were the anti-HLA-E antibody 3D12 which binds to correctly folded HLA-E regardless of peptide, and monoclonal antibody 19B6, which binds to peptide RMAPRTLVL cross-linked to HLA-E and not to the non-cross-linked form. The graphs show flow cytometry plots where the y axis represents the number of cells and the x axis is the fluorescent intensity of fluorochrome-labelled antibodies with the specificities indicated.

FIG. 3—shows a diagrammatic representation of a mutation of position 45 in the B pocket of HLA-E. The methionine at position 2 in the peptide (RMYSPTSIL) is closest to the end carbon of HLA-E methionine M45, distance 3.4-3.6 A, shown by PDB coordinates and Pymol (https://pymol.org/2/). This requires a long free thiol linker to create a disulphide bond with position 2 in the peptide.

FIG. 4—demonstrates that HLA-E with cysteine at position 45 stably refolds with the Signal VL9 (VMAPRTLVL) peptide. The peak at the 60 ml elution fraction represents refolded HLA-E-β2micrglobulin-peptide after the refolding reaction, showing a high yield of correctly folded protein.

FIG. 5—Images (i), (ii) and (iii) show the crystal structure of the B pocket with the Mtb44 peptide RLPAKAPLL substituted at position 2 with glutamine, phenyl-alanine or leucine. These changes had no effect on T cell recognition of the peptide (Walters et al Nat Comms 2018). The synthetic amino acid 2-amino-5-mercaptopentanoic acid (PubChem CID 10419348) SH-CH2-CH2-CH2-CHNH2-COOH has a suitable chain length to form a disulphide bridge between position 2 of a peptide antigen and cysteine 45 of HLA-E.

FIG. 6—demonstrates a chemical pathway for the production of a homocysteine analogue (2S)-2-amino-5-sulfanylpentanoic acid using enzyme resolution.

FIG. 7—Demonstrates thermal gain of HLA-EC84-C139 over canonical HLA-E when incubated with 100M excess peptide. 10 uM of pre-refolded HLA-E and HLA-EC84-C139 material was incubated with 100M excess test peptides (P1-P9) for 30 minutes at room temperature prior to thermal melt analysis using a Prometheus NT.48 Series Differential Scanning Fluorimetry instrument. Test samples were split between two Prometheus NT.48 Series nanoDSF Grade Standard Capillaries and a ramp rate of 1° C./min from 20° C. to 95° C. was applied. The ratio for fluorescence emission at 330 nm and 350 nm was used to derive the thermal melt of unfolding (Tm). Shown are the relative Tm data for canonical HLA-E (grey) and HLA-EC84-C139 (red) datasets, where the corresponding no-peptide control Tm data for canonical HLA-E and HLA-EC84-C139 have been subtracted, respectively. The numbers plotted above the red bars denote the equivalent Tm loss/gains obtained for the HLA-EC84-C139 variant over canonical HLA-E.

FIG. 8—Demonstrates thermal gain of HLA-E^{S147W} over canonical HLA-E when incubated with 100M excess peptide. 10 uM of pre-refolded HLA-E or HLA-E^{S147W} material was incubated with 100M excess test peptides (P1-P9) for 30 minutes at room temperature prior to thermal melt analysis using a Prometheus NT.48 Series Differential Scanning Fluorimetry instrument. Test samples were split between two Prometheus NT.48 Series nanoDSF Grade Standard Capillaries and a ramp rate of 1° C./min from 20° C. to 95° C. was applied. The ratio for fluorescence emission at 330 nm and 350 nm was used to derive the thermal melt of unfolding (Tm). Shown are the relative control Tm gains for canonical HLA-E (right) and HLA-E^{S147W} (left) datasets, where the corresponding no-peptide control Tm data for canonical HLA-E and HLA-E^{S147W} have been subtracted, respectively. The numbers plotted above the red bars denote the equivalent Tm loss/gains obtained for the HLA-E^{S147W} variant over canonical HLA-E.

FIG. 9—Thermal gain of HLA-E^{H99Y}, HLA-E^{F116Y} and HLA-E^{S147W} over canonical HLA-E when incubated with 10M excess peptide

10 uM of pre-refolded HLA-E and HLA-E^{H99Y} (A), HLA-E^{F116Y} (B), or HLA-E^{S147W} (C) material was incubated with 10M excess test peptides (from panel pA to pG) for 30 minutes at room temperature prior to thermal melt analysis using a Prometheus NT.48 Series Differential Scanning Fluorimetry instrument. Test samples were split between two Prometheus NT.48 Series nanoDSF Grade Standard Capillaries and a ramp rate of 1° C./min from 20° C. to 95° C. was applied. The ratio for fluorescence emission at 330 nm and 350 nm was used to derive the thermal melt of unfolding (Tm). Shown are the relative control Tm gains for canonical HLA-E (left columns) and HLA-E^{H99Y}, HLA-^{F116Y} or HLA-E^{S147W} (right columns) datasets, where the corresponding no-peptide control Tm data for canonical HLA-E and HLA-E^{H99Y}, HLA-E^{F116Y} or HLA-ES147W have been subtracted, respectively. The numbers plotted above the right-hand columns denote the equivalent Tm gains obtained for the HLA-E^{H99Y}, HLA-E^{F116Y} or HLA-E^{S147W} variants over canonical HLA-E, respectively.

### MATERIALS AND METHODS

Refolding HLA-E(M45C): β2-Microglobulin in Urea-Mes, at a final concentration of 2 μM, was refolded at 4° C. for 30 min in a macro-refolding buffer prepared in MiliQ water containing 100 mM Tris, pH 8.0, 400 mM L-arginine monohydrochloride, 2 mM EDTA, 5 mM reduced glutathione, and 0.5 mM oxidized Glutathione. The UV-sensitive peptide VMAPRTLVL was subsequently added to the β2M refold to achieve a peptide concentration of 30 μM. HLA-E*01:03(M45C) heavy chain was subsequently pulsed into the refolding buffer until a final concentration of 1 μM was reached. Following incubation for 72 h at 4° C., HLA-E refolds were filtered through 1.0 μm cellular nitrate membranes to remove aggregates prior to concentration by the VivaFlow 50R system with a 10 kDa molecular weight cut-off (Sartorius) and subsequent concentration in 10 kDa cut-off VivaSpin Turbo Ultrafiltration centrifugal devices (Sartorius). Samples were then separated according to size into 20 mM Tris pH8, 100 mM NaCl by fast protein liquid chromatography (FPLC) on an AKTA Start System using a Superdex S75 16/60 column. Elution profiles were visualized by UV absorbance at 280 mAU, enabling differentiation of correctly refolded HLA-E-β2M-pepide complexes from smaller non-associated β2M and larger misfolded aggregates. FPLC-eluted protein peak fractions were combined and concentrated to 3 mg/mL using 10 kDa cut-off VivaSpin Turbo Ultrafiltration centrifugal devices prior to use in the HLA-E peptide exchange ELISA-based assay. One microliter aliquots were analyzed by non-reducing SDS-PAGE electrophoresis on NuPAGE™ 12% Bis-Tris protein gels (ThermoFisher Scientific) to confirm the presence of non-aggregated HLA-E heavy chain and β2m.

### Thermal Melt Assay

By ROX Dye Incorporation: The thermostability of canonically refolded HLA-E-β2m peptide complexes and C terminus extended peptides with a cysteine refolded with HLA-E containing a tyrosine to cysteine mutation was determined by heat-induced fluorescent dye incorporation, using the commercially available Protein Thermal Shift Dye Kit™ (Applied Biosystems). 5 μg of test HLA-E-β2m complexes was aliquoted into 0.1 mL MicroAmp Fast Optical 96-well plates containing pre-mixed Protein Thermal Shift Dye and Protein Thermal Shift Buffer. Sample buffer (either PBS or Tris pH8, 100 mM NaCL) was added to achieve a final volume of 20 μL. Control samples reconstituted with buffer were prepared to monitor background fluorescent signal. Both samples and controls were set up in quadruplicate. Thermal-driven dye incorporation was measured on an Applied Biosystem Real-Time 7500 Fast PCR System. Data was collected over a temperature ramp ranging from 25 to 95° C., with 1° C. intervals. Melt curve data were analysed using Protein thermal Shift Software v1.3, and median Derivative Tm values (° C.) are reported.

By Differential Scanning Fluorimetry: The thermal stability of canonical HLA-E and HLA-E H99Y, HLA-E F116Y and HLA-E S147W material was measured by Differential scanning fluorimetry analysis. The individual HLA-E proteins were incubated at 0.45 mg/ml with Molar excess of peptide in 20 mM Tris pH7, 100 mM NaCl buffer at a final volume of 20 uL. Following a 30 minute incubation at room temperature, individual samples were subsequently split, transferred into two Prometheus NT.48 Series nanoDSF Grade Standard Capillaries (Nanotemper, Munich, Germany) and then placed in the capillary tray of a Prometheus NT.48 fluorimeter (Nanotemper). Excitation power was pre-adjusted to obtain between 8000 and 20,000 Raw Fluorescence Units for fluorescence emission at 330 nm and 350 nm. A thermal ramp ranging from 20° C. to 95° C., at a rate of 1° C./min, was applied. Automated thermal melt data calling was generated by the analysis software within PR.ThermControl, (version 2.1.5) software.

Single chain HLA-E construct assays: The single chain HLA-E-β2m-peptide constructs contained the coding sequence of the mature form of HLA-E*0103. The tyrosine at position 84 was mutated to alanine by overlap extension PCR and the fragment was inserted into pEGFP-N1 using BamH I, downstream of a Hind III-BamH I cassette that comprised the signal sequence of HLA-E*01:01, sequence encoding the required peptide, a flexible glycine-serine linker ([GGGGS]3), the coding sequence of the mature form of β2-microglobulin, and a second flexible linker ([GGGGS]4).

HEK 293T cells were maintained between 10% and 90% confluency at 37° C./5% CO₂ in DMEM (Life Technologies) supplemented with 10% Fetal Bovine Serum (SeraLabs), and Penicillin/Streptomycin (50 units/ml and 50 μg/ml, respectively; Life Technologies). Transfections were carried out in 6-well plates using GeneJuice (Millipore) as per the manufacturer's instructions. One million 293T cells were stained in 100 μl of PBS at 4° C. for 15 minutes with primary antibody (3D12 for HLA-E [BioLegend], washed twice with PBS, stained with secondary antibody (allophycocyanin-crosslinked Goat-Anti-Mouse (H+L) F(ab′)2 fragment [Life Technologies]), washed as before, and fixed in 100 μl of Cytofix (BD Biosciences). Stained cells were acquired using a CyAn ADP Analyser (Beckman Coulter), and analysed using FlowJo (Tree Star).

### Synthesis of Fmoc- and trityl-protected 2-amino-5-sulfanylpentanoic Acid for Peptide Synthesis

### Synthesis of (2S)-2-(9H-fluoren-9-ylmethoxycarbonylamino)-5-(tritylthio) Pentanoic Acid

### Synthesis of diethyl 2-(3-bromopropyl)-2-acetamidomalonate (Compound 1)

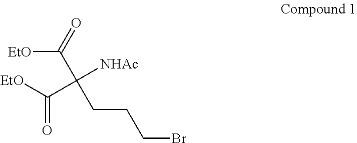 [SMILES: CCOC(=O)C(CCCBr)(NC(C)=O)C(=O)OCC]

The method for synthesising compound 1 was based on the publication by Yavuz et al. (https://doi.org/10.1002/ardp.201700185). Diethyl acetamidomalonate (2.17 g, 10 mmol, 1.0 eq) was added in portions to an ice-cooled solution of sodium hydride (60% suspension in oil, 1.0 eq) in dry DMF (10 ml). The mixture was stirred at room temperature for 2 hours and then 1,3-dibromopropane (4.1 ml, 40 mmol, 4.0 eq) was added. The reaction was allowed to proceed at room temperature for 24 hours, and TLC was used to confirm that the reaction was complete. The reaction mixture was poured into iced water (50 ml) and extracted with ethyl acetate (3×50 ml). The organic phase was dried with sodium sulfate, and then concentrated under reduced pressure. Compound 1 was purified by flash chromatography with an eluent of hexane/ethyl acetate 3:1.

### Synthesis of diethyl 2-(3-tritylthiopropyl)-2-acetamidomalonate (Compound 2)

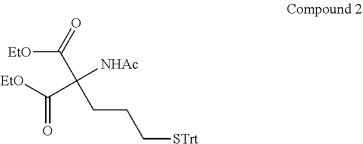

Triphenylmethane thiol (2.75 g, 10 mmol, 1.0 eq) was added in portions to an ice-cooled solution of sodium hydride (60% suspension in oil, 1.0 eq) in dry DMF (10 ml). After 30 minutes, a solution of 1 (3.38 g, 10 mmol, 1.0 eq) in dry DMF (10 ml) was added dropwise with stirring. The reaction was allowed to reach room temperature and stirred for 24 hours. TLC was used to confirm the reaction was complete. The reaction mixture was poured into iced water (50 ml) and extracted with ethyl acetate (3×50 ml). The organic phase was dried with sodium sulfate, and then concentrated under reduced pressure. Compound 2 was purified by flash chromatography.

### Synthesis of 2-acetamido-5-tritylthiopentanoic Acid (Compound 3)

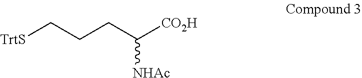

Sodium hydroxide (1.0 g, 25 mmol, 2.5 eq) was dissolved in water (100 ml). Compound 2 (5.34 g, 10 mmol, 1.0 eq) was added and the solution was heated to reflux for 24 hours. LC-MS was used to verify that the reaction had completed. The solution was acidified using 2M HCl. Ethyl acetate (4×50 ml) was used to extract the compound and dried with sodium sulfate. Solvent was removed under reduced pressure and compound 3 was purified using flash chromatography if necessary.

### Synthesis of (2S)-2-amino-5-(tritylthio)pentanoic Acid (Compound 4)

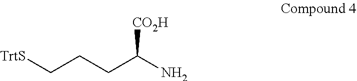

This method for N-acetyl rac-amino acid resolution was based on the publication by Gharakhanian et al. (https://doi.org/10.1021/jacs.9b07223). Compound 3 (4.33 g, 10 mmol, 1.0 eq) was dissolved in water (100 ml) and the pH of the solution was adjusted to 7.5-8.0 using 1M potassium hydroxide. Porcine kidney acylase I (1100 U/mg protein; 4.2 U acylase per mmol of N-acetyl amino acid) was added and the reaction mixture was stirred for 24 hours. The mixture was then heated to 60° C. and activated charcoal was added (2.5 ml). The mixture was then filtered and the filtrate was adjusted to pH 1.5 with 2M HCl. The solution was washed with ethyl acetate (3×50 ml) and the aqueous layer applied to a column of Dowex-50 (H⁺). Water was added to column until becoming neutral. The L-amino acid was eluted with 1N aqueous ammonia, and the solvent removed by lyophilisation.

### Synthesis of (2S)-2-(9H-fluoren-9-ylmethoxycarbonylamino)-5-(tritylthio)pentanoic Acid 5

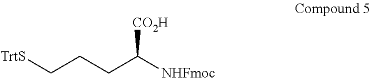

Compound 4 (3.91 g, 10 mmol, 1.0 eq) was dissolved in 9% sodium hydrogencarbonate solution (40 ml), and the solution cooled to 0° C. in an ice bath. Fmoc-OSu (6.74 g, 20 mmol, 2.0 eq) in DMF (40 ml) was added to the solution and the reaction was allowed to proceed for 2 hours. The reaction was acidified using 2M HCl and was then extracted with ethyl acetate (3×50 ml), and dried with sodium sulfate. Solvent was removed under reduced pressure and compound 5 was purified using flash chromatography if necessary.

### EXAMPLES

### HLA-E Expression

HLA-E protein can be made using cells, cell lines or by transfecting DNA plasmids encoding HLA-E into bacteria or fungal cells (O'Callaghan et al. Production, crystallization, and preliminary X-ray analysis of the human MHC class Ib molecule HLA-E. Protein Sci. 1998; 7(5):1264-6; O'Callaghan et al., Structural features impose tight peptide binding specificity in the nonclassical MHC molecule HLA-E. Mol Cell. 1998; 1(4):531-41). The protein can then be tested for binding potential antigenic peptides in a refolding binding assay. As described by Walters et al (Walters et al., Detailed and atypical HLA-E peptide binding motifs revealed by a novel peptide exchange binding assay. Eur J Immunol. 2020), HLA-E can be refolded with VL9 peptide that contains an ultraviolet light sensitive amino acid at position 5 in the sequence VMAPJTLVL where J is 3-amino-3-(2-nitrophenyl)-propionic acid. In the presence of test peptides, the folded HLA-E is then exposed to light and VMAPJTLVL is cleaved and then can be replaced by a test peptide if the latter binds. The correctly folded HLA-E is then tested in a sandwich ELISA assay, where the first antibody is anti-HLA-E (3D12) and then the second test antibody is anti-β2 microglobulin. Peptides that give a binding signal that is >20% of that given by VL9 are considered to be binders. (Walters et al., Detailed and atypical HLA-E peptide binding motifs revealed by a novel peptide exchange binding assay. Eur J Immunol. 2020).

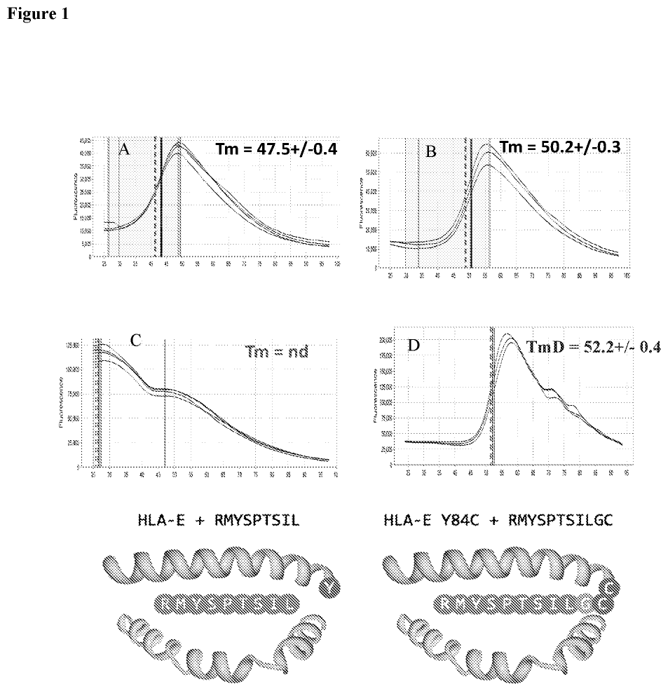

However, many of those peptides bind unstably as seen in blue native gels, with a diffuse rather than compact peak (Walters et al., Pathogen-derived HLA-E bound epitopes reveal broad primary anchor pocket tolerability and conformationally malleable peptide binding. Nat Commun. 2018; 9(1):3137) and by low or unobtainable thermal melt Tm (Walters et al., Pathogen-derived HLA-E bound epitopes reveal broad primary anchor pocket tolerability and conformationally malleable peptide binding. Nat Commun. 2018; 9(1):3137), suggesting rapid dissociation (FIG. 1). Nevertheless, these are often immunodominant epitopes when T cell responses are present during natural or experimental infection for example in mycobacteria or rhesus cytomegalovirus strain 68-1 infection) (Walters et al 2020). Further, several immunodominant epitopes are seen as non-binders in these assays.

In a second assay, the peptide sequence is encoded in a synthetic oligonucleotide which is coupled to cDNA sequences expressing β2m and the HLA-E heavy chain, with or without a peptide sequence, separated by linker sequences and under a CMV promoter. This DNA is then transfected into a cell line, such as HEK 293T cells, and the ability of the transfected single chain construct to come to the cell surface is measured by antibody staining in a flow cytometer (Hansen et al., Broadly targeted CD8(+) T cell responses restricted by major histocompatibility complex E. Science. 2016; 351(6274):714-20). When the DNA-encoded peptide sequence binds to HLA-E there is surface expression which is proportional to the binding strength of the peptide. This assay gives results comparable to the ELISA assay.

As discussed previously, for peptide:HLA-E specific antibody and T cell/TCR selection and immunisation, it is only possible to use stable protein complexes and cells transfected with stable single chain trimers. The invention therefore allows such activity.

### Discussion

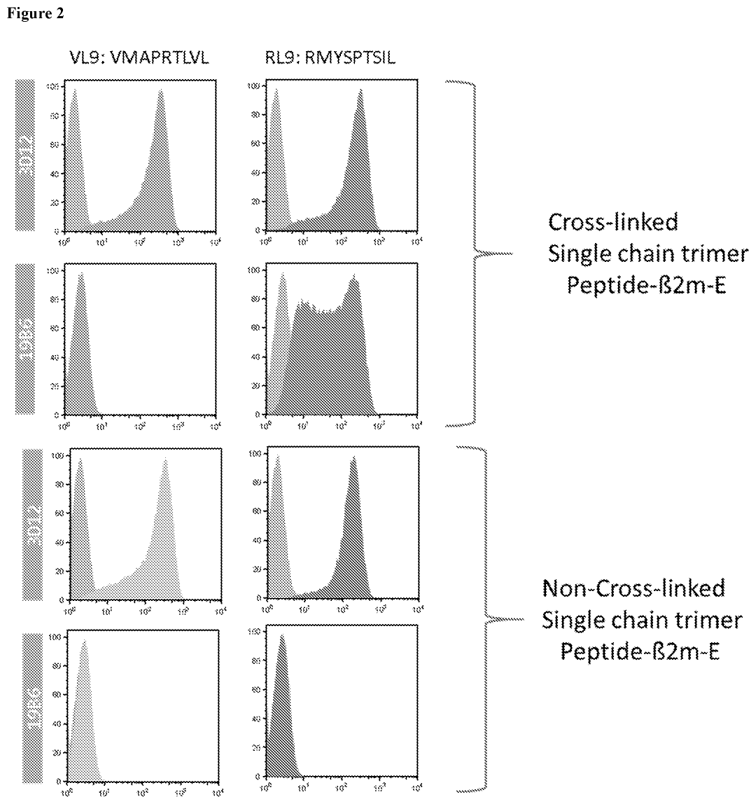

When the method of Fremont et al. (Structural engineering of pMHC reagents for T cell vaccines and diagnostics, Chem Biol, 2007 August; 14(8):909-22) was used to stabilise binding of the relatively low affinity HIV-1-Gag epitope peptide RMYSPTSIL to HLA-E, the binding was stabilised and a clean melting temperature (Tm) was obtained. This peptide was then used to immunise mice transgenic for HLA-B27-human β2m, and the monoclonal antibody 19B6 was obtained that was specific for the cross-linked peptide HLA-E immunogen. However, when tested on cells that expressed HLA-E and β2m and pulsed with the non-cross-linked peptide RMYSPTSIL, no binding was seen (FIG. 2), therefore demonstrating that the method causes some conformational change to the natural complex.

More specifically demonstrated by FIG. 2, monoclonal antibody 19B6 is specific for the HIV-1 Gag RL9 peptide RMYSPTSIL cross linked to HLA-E through a disulphide bridge between a cysteine mutated into HLA-E at position 84 and a cysteine added as a glycine-cysteine (GC) dipeptide added to the C terminus of RL9, giving the peptide RMYSPTSILGC. The HLA-E—cross linked to RL9 was expressed as a single chain trimer encoded by plasmid DNA transfected into the cell line HEK293T. 19B6 detected the HLA-RL9 complex at the surface of these cells, as did the 3D12 antibody which is specific for HLA-E regardless of the peptide bound. The 19B6 antibody did not bind to the signal VL9 peptide VMAPRTLVL cross linked in the same manner. However the antibody 19B6 does not recognise HLA-E-RL9 expressed as a single chain trimer in transfected HEK293T cells without the dicysteine bridge, whereas antibody 3D12 does recognise HLA-E on the surface of these cells, indicating that the RL9 peptide was present, as a bound peptide is known to be necessary for HLA-E surface expression. Therefore, the cross-linking of RL9 is likely to have introduced a conformational change in the structure of HLA-E-RL9 which is seen by the 19B8 antibody, and is absent in HLA-E bound to non-cross linked RL9.

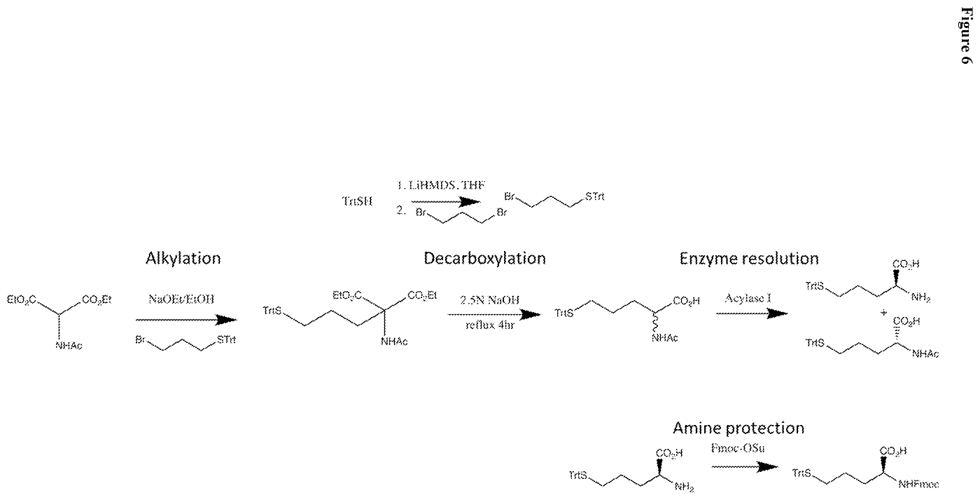

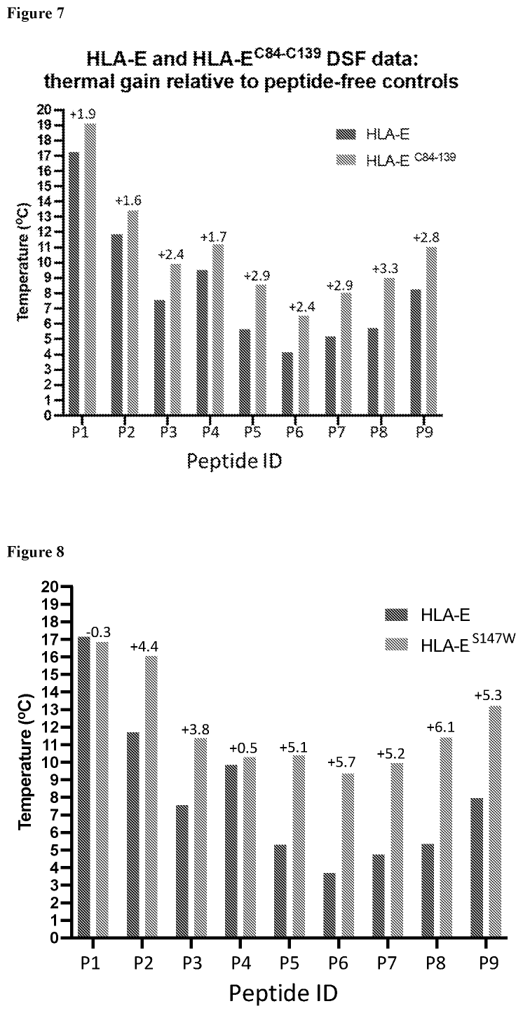

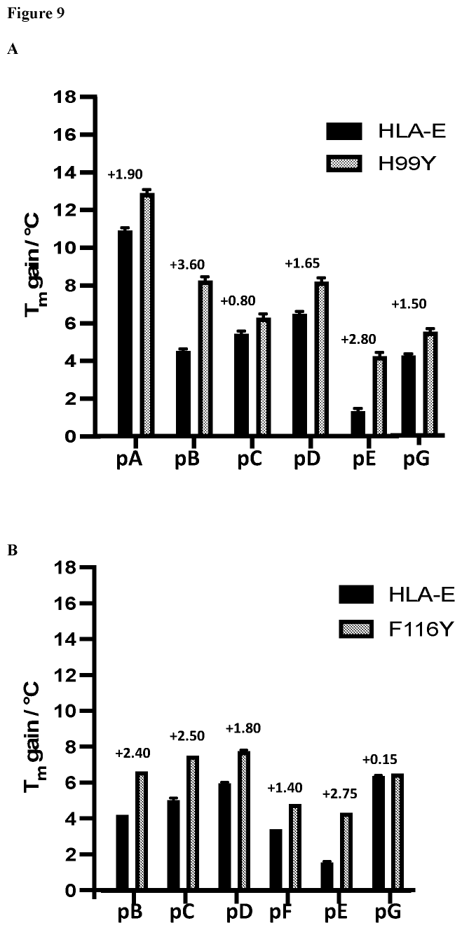

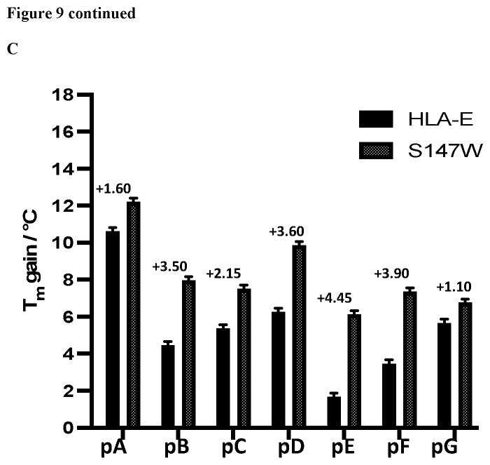

The inventors demonstrate herein that using the invention described herein HLA-E can be mutated to bind peptide antigens more stably. For example, it is demonstrated that when the methionine at position 45 in the deep B pocket in the HLA-E heavy chain is mutated to cysteine the heavy chain still refolds well with the canonical VL9 peptide (FIG. 5). The B pocket is spacious and can accommodate side chains methionine, leucine, glutamine and phenylalanine at position 2 of the peptide and crystallise (FIG. 5) without altering peptide presentation to T cells. Therefore, the peptides VL9 and RL9, as examples, with methionine replaced with a sulphydryl (SH) containing synthetic peptide will form a covalent disulphide (S-S) bridge with a mutant HLA-E as described herein. This will stabilise the peptide-HLA-E interaction at a conformationally stable portion of the HLA-E structure, distally away from the less stable interaction between the HLA-E structure and the C terminal region of the peptide.

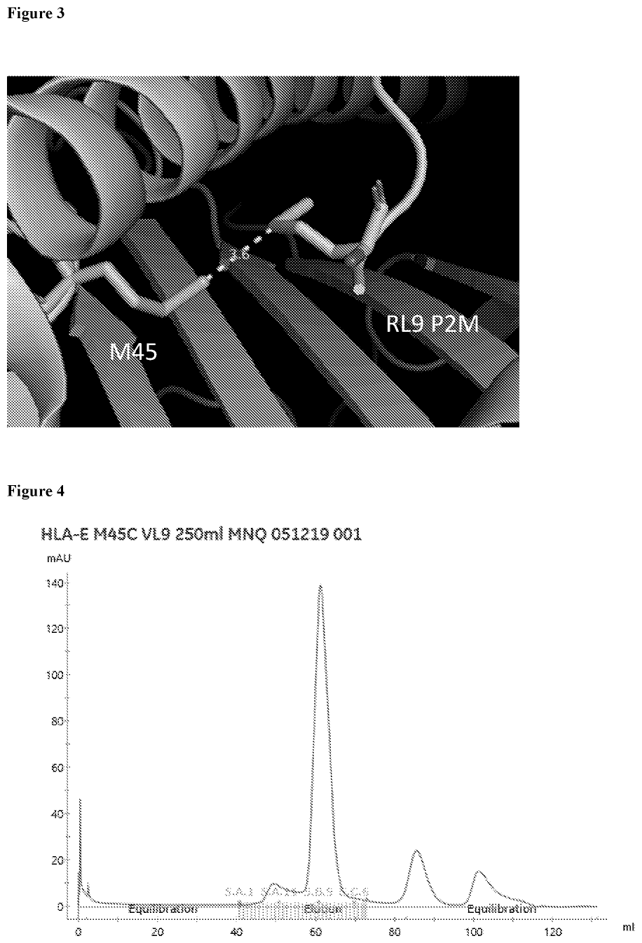

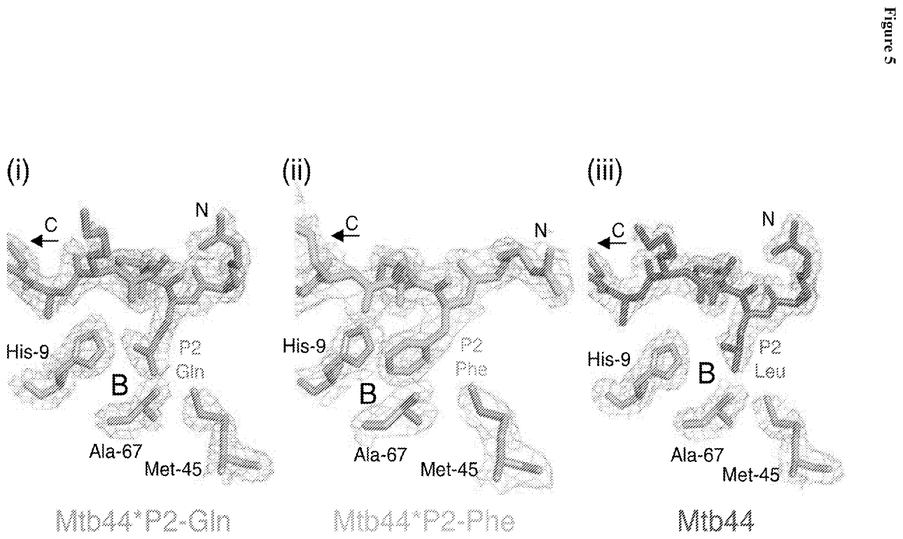

A synthetic amino acid, such as 2-Amino-5-mercaptopentanoic acid (synonym: 2-Amino-50-sulfanylpentanoic acid) (PubChem CID 10419348) may be used to allow the crosslink to form and bridge the gap between the two —SH groups which are spatially distant, at 3.6 Å (FIG. 3). This synthetic amino acid, Fmoc and Trityl protected for peptide synthesis, will be incorporated into the peptides, for example into VJAPRTLV and RJYSPTSIL, where J is the synthetic amino acid. The peptides will then be used to refold HLA-E, with and without the M45C mutation, together with β2microglobulin. The stability of the complex will then be tested by blue native gel analysis and thermal melting point (Tm) determination. Finally the refolded HLA-E molecule will be biotinylated using the Bir A enzyme at the C terminus to form HLA-E tetramers. These will then be tested for reactivity with T cell clones specific for HLA-E and the RL9 peptide and with natural killer cells expressing the NKG2A-CD94 receptor which is specific for HLA-E and the canonical VL9 peptide.

Other synthetic SH-containing amino acids, including 2-amino-6-sulfanylhexanoic acid at position 2 in the peptide may also be used with HLA-E M45C or other mutant HLA-E heavy chains of the invention, such as HLA-E Y7C, which is at the mouth of the B pocket and also contributes to the A pocket. This provides the option of introducing a homocysteine at position 2 in the peptide, which can be crosslinked with the mutant HLA-E.

The inventors have also mutated tyrosine 171 of HLA-E to cysteine in the A pocket, which can be cross linked to a mercaptopropionic acid at the N-terminus of the peptide, such as in position 1 or position 2 of the peptide.

Such combinations of mutant HLA-E and peptide provide an environment which will be sterically favourable to form disulphide bonds, and therefore stabilise complex formation in a manner which retains the conformation of the natural complex.

1. A mutant HLA-E heavy chain comprising one or more mutation which permits the formation of a HLA-E:peptide complex with increased stability when compared to the complex without the mutant HLA-E heavy chain.

2. The mutant HLA-E heavy chain of claim 1, wherein the mutant HLA-E heavy chain is capable of being crosslinked to a peptide antigen, optionally wherein the crosslink is between one of the mutations in the HLA-E heavy chain and the peptide antigen.

3. The mutant HLA-E heavy chain of claim 2, wherein crosslinking introduces a covalent bond between an amino acid in the mutant HLA-E heavy chain and an amino acid in the peptide antigen, optionally wherein the covalent bond is a disulphide bond.

4. The mutant HLA-E heavy chain of any of claims 1-3, wherein the mutant HLA-E heavy chain is derived from human HLA-E of SEQ ID NO:1, SEQ ID NO: 2, SEQ ID NO: 3, or SEQ ID NO: 4.

5. The mutant HLA-E heavy chain of any of claims 1-4, wherein the one or more mutation in the HLA-E heavy chain is in the A pocket, or the B pocket.

6. The mutant HLA-E heavy chain of any of claims 1-5, wherein:
a) the one or more mutation is of an amino acid at one or more of position 28, 80, 84, 98, 184, 189, or 192 of SEQ ID NO: 1 or SEQ ID NO: 3, or one or more amino acid at a position equivalent thereto of SEQ ID NO: 1 or SEQ ID NO: 3; or
b) at least the amino acids at positions 28 and 192 of SEQ ID NO: 1 or SEQ ID NO: 3, or the amino acids at positions equivalent thereto of SEQ ID NO: 1 or SEQ ID NO: 3 are mutated; or
c) the one or more mutation is of an amino acid at one or more of position 7, 59, 63, 77, 163, 167, or 171 of SEQ ID NO: 2 or SEQ ID NO: 4, or one or more amino acid at a position equivalent thereto of SEQ ID NO: 2 or SEQ ID NO: 4; or
d) at least the amino acids at positions 7 and 171 of SEQ ID NO: 2 or SEQ ID NO: 4, or at positions equivalent thereto of SEQ ID NO:2 or SEQ ID NO: 4 are mutated; or
e) the one or more mutation is of an amino acid at one or more of position 28, 30, 66, 84, 87, 88, or 91 of SEQ ID NO: 1 or SEQ ID NO: 3, or one or more amino acid at a position equivalent thereto of SEQ ID NO: 1 or SEQ ID NO: 3; or
f) at least the amino acid at position 66 of SEQ ID NO: 1 or SEQ ID NO: 3, or the amino acid at a position equivalent thereto of SEQ ID NO: 1 or SEQ ID NO: 3 is mutated;
g) the one or more mutation is of an amino acid at one or more of position 7, 9, 45, 63, 66, 67, or 70 of SEQ ID NO: 2 or SEQ ID NO: 4, or one or more amino acid at a position equivalent thereto of SEQ ID NO: 2 or SEQ ID NO: 4; or
h) at least the amino acid at position 45 of SEQ ID NO: 2 or SEQ ID NO: 4, or the amino acid at a position equivalent thereto of SEQ ID NO: 2 or SEQ ID NO: 4 is mutated; or
i) the one or more mutation is of an amino acid at one or more of position 99, 116 or 147 of SEQ ID NO: 2 or SEQ ID NO: 4, or one or more amino acid at a position equivalent thereto of SEQ ID NO: 2 or SEQ ID NO: 4.

7. The mutant HLA-E heavy chain of any of claims 1-6, wherein the mutant HLA-E heavy chain comprises or consists of a sequence of SEQ ID NO: 5, SEQ ID NO: 6, SEQ ID NO: 7 or SEQ ID NO: 8.

8. The mutant HLA-E heavy chain of any of claims 1-7, wherein the one or more mutation is to one or more amino acid with a free sulphydryl group, such as a cysteine.

9. A peptide which is capable of crosslinking to the mutant HLA-E heavy chain of any of claims 1-8.

10. The peptide of claim 9 wherein the peptide is about 9 to about 11 amino acids long.

11. The peptide of any of claims 9-10, wherein the amino acid in the first or second position is capable of crosslinking to the mutant HLA-E heavy chain of any of claims 1-8.

12. The peptide of any of claims 9 to 11, wherein the amino acid in the first or second position is a cysteine, a homocysteine, or a synthetic amino acid comprising a free sulphydryl group.

13. The peptide of claim 12, wherein the synthetic amino acid is a homocysteine analogue, (2S)-2-amino-5-sulfanylpentanoic acid or (2S)-2-amino-6-sulfanylhexanoic acid.

14. A protein complex comprising or consisting of the mutant HLA-E heavy chain of any of claims 1-8, and the peptide of any of claims 9-13.

15. The protein complex of claim 14, wherein the mutant HLA-E heavy chain and peptide are crosslinked, optionally wherein the crosslink is via a disulphide bond between a mutant amino acid in the HLA-E heavy chain and the amino acid at the first or second position in the peptide.

16. The protein complex of claim 14 or 15, further comprising β2 microglobulin.

17. The protein complex of complex of any of claims 14 to 16 wherein the complex is more stable than the complex formed between an unmutated HLA-E heavy chain and the peptide of any of claims 9 to 13.

18. A polypeptide comprising one or more, or all of: a sequence of the mutant HLA-E heavy chain of any of claims 1-8, the peptide of any of claims 9-13, and β2 microglobulin,
optionally wherein the sequence of the mutant HLA-E heavy chain is separated from the sequence of the peptide and/or β2 microglobulin via a linker sequence;
optionally wherein the sequence of the peptide is separated from the sequence of the mutant HLA-E heavy chain and/or β2 microglobulin via a linker sequence.

19. A method of identifying antigen binding polypeptides which recognise a HLA-E:peptide complex, wherein the method comprises:
a) crosslinking the peptide of any of claims 9-13 to the mutant HLA-E heavy chain of any of claims 1-8, to form a crosslinked HLA-E:peptide complex; and
b) screening for antigen binding polypeptides which recognise the crosslinked HLA-E:peptide complex [symbol] optionally wherein the crosslinking is covalent, for example via disulphide bonding, and optionally wherein the antigen binding polypeptide is an antibody or a T-cell receptor (TCR).

20. A method of increasing the stability of an HLA-E:peptide complex, wherein the method comprises crosslinking the peptide of any of claims 9-13 to the mutant HLA-E heavy chain of any of claims 1-8, such that the HLA-E:peptide complex is stabilised, when compared to a complex of the non-mutated peptide and a non-mutated HLA-E heavy chain, optionally wherein the crosslinking is covalent, for example via disulphide bonding.

21. A nucleic acid encoding one or more of the mutant HLA-E heavy chain of any of claims 1-8, the peptide of any of claims 9-13, the protein complex of any of claims 14-17 or the polypeptide of claim 18.
